(19) United States
(12) Patent Application Publication
(10) Pub. No.: US 2022/0296772 A1
(43) Pub. Date: Sep. 22, 2022

(54) NOVEL GLUE FOR EMBOLIZATION OF LYMPHATIC LEAKAGE

(71) Applicants: Ghassan S. Kassab, La Jolla, CA (US); Ali Dabiri, La Jolla, CA (US); Niki Bayat, Los Angeles, CA (US); Roby Menefee, Los Angeles, CA (US)

(72) Inventors: Ghassan S. Kassab, La Jolla, CA (US); Ali Dabiri, La Jolla, CA (US); Niki Bayat, Los Angeles, CA (US); Roby Menefee, Los Angeles, CA (US)

(21) Appl. No.: 17/373,714

(22) Filed: Jul. 12, 2021

Related U.S. Application Data
(60) Provisional application No. 63/050,150, filed on Jul. 10, 2020.

(51) Int. Cl. A61L 24/00; A61L 24/02; A61L 24/06

(52) U.S. Cl. CPC A61L 24/0031; A61L 24/02; A61L 24/06; A61L 2400/06; A61L 2430/36; A61L 2300/102

(57) ABSTRACT

A novel glue for embolization of lymphatic leakage. An optimized lymphatic embolization agent (LEA) as described herein comprises a NAM hydrogel and tantalum at a mixture of at or about 1:3 tantalum to NAM hydrogel, wherein said LEA is radiopaque.

### PRIORITY

The present application is related to, and claims the priority benefit of, U.S. Provisional Patent Application Ser. No. 63/050,150, filed Jul. 10, 2020, the contents of which are incorporated herein directly and by reference in their entirety.

### BACKGROUND

The lymphatic system is responsible for recycling, immune, and waste functions in the body, and hence it is critical to long term homeostasis. Lymph node dissections, transplants, vessel reconstructions, and other surgical procedures may inadvertently damage lymphatic channels. The subsequent leakage of lymphatic fluid, a combination of white blood cells, proteins, and fats collected from interstitial tissues, can cause serious injury when left untreated. While less fluid flows through lymphatic vessels than blood vessels, several liters still flow through the lymphatic system daily. This extensive network draws from blind-ended lymphatic capillaries and flows through a network of vessels and organs, crossing important, high-volume, regions like the cisterna chyli and thoracic duct to end at the subclavian veins.

Although lymphatic complications are rare, they require urgent medical attention. Such complications come in two forms: Lymphatic stasis and lymphatic leakage. The inventive disclosure referenced herein addresses the development of an effective treatment for the latter. Exemplary leaks include, but are not limited to, chylous ascites, lymphocele, lymphorrhea, chylorrhea, chyloretroperitoneum, chylothorax, protein losing enteropathy, plastic bronchitis, and more. Loss of fluid, triglyceride, lymphocyte, and immunoglobulin at leakage sites can lead to dehydration, nutritional deficiency, and immunologic dysfunction. Lymphatic leakage also increases susceptibility to infection, causing a cascade of harm if leaks go untreated. Compression of vital structures can also occur in chylous ascites, lymphocele, and chylothorax due to increased tissue pressures. At the very least, patients with lymphatic leakage can expect pain and a prolonged hospital stay (about 12 to 20 days).

Due to a high success rate, and minimal complications, thoracic duct embolization is becoming a common intervention to treat lymphatic leakage. Existing embolization materials offer poor control over delivery windows, have a range of chemical and material adverse effects, and are available only at relatively high costs.

Lymphatic leakage treatment protocols are variable with delays in intervention ranging from several weeks to 2 months. In some cases, 66% of patients for one study, fasting and medical treatment were sufficient to treat lymph disorders like small fistulas. Conversely, some investigators believe early surgical intervention should be first action for lymphatic leakage to control the leakage site, avoid complications and shorten hospitalization. Mortality rates for delayed treatment methods can reach 50% while those for surgical intervention are only 10%. Surgical approaches include peritoneovenous shunt, lymphostasis by suture ligation or embolization agent, and surgery combined with sealant. While peritoneovenous shunt and operation under direct vision are the most common treatments, improved outcomes are driving the adoption of lymphatic embolization.

Thoracic duct embolization, as referenced above, is a standard treatment for compromised lymph vessels that involves the delivery of an occlusive sealant via catheter. One method of lymphatic embolization is when a glue, such as N-butyl cyanoacrylate (NBCA) glue, is delivered into lymphatic vessel-filled tissue to treat downstream lymphatic leaks. Several studies describe the efficacy of this glue when treating post-surgical pelvic lymphoceles, groin lymphorrhea, abdominal ascites, and other disease states. Accessing the vessels via a lymph node, these procedures use catheter injection to fill a target region with glue, normally about 1 ml. Though simplistic, this procedure has shown 100% clinical success for studies across various use cases. In one study, lymphatic leaks resolved in 8 of 10 (80%) patients and median time to resolution was 7 days (with a range of 1-17 days), showing the possibility for a quick recovery when such a non-invasive procedure is used. Patients occasionally suffer recurrence or unsuccessful embolization, requiring laparoscopic follow up procedures. Nevertheless, the simplicity of the operation has made it a desirable alternative to other surgical methods. Most studies also report few complications, further demonstrating the reliability of embolization as a treatment for lymphatic disorders.

As early as 2002, lymphatic embolization with glues was documented as a lower risk operation to treat lymphatic leaks. Dr. Constantine Cope, the interventional radiologist and lymphangiographer who invented the lymphatic embolization procedure with coils only a few years prior, anticipated the utility of various occlusive agents when there were very limited options. At that time, detachable coils, N-Butyl Cyanoacrylate (NBCA) glue, and Onyx® (a liquid polymerizing embolic agent consisting of ethylene copolymer and vinyl alcohol dissolved in dimethyl sulfoxide (DMSO)) were novel and exciting materials used primarily for the treatment of arteriovenous malformations or intracranial aneurysms, disorders of the circulatory system. Detachable coils are generally formed from platinum and have been modified in various ways since their invention to improve their safety profile. The associated risk of migration, vessel rupture, and perforation spurred modification with softer components, but also demonstrated the need for materials that provide a different form of mechanical occlusion. N-Butyl Cyanoacrylate (Trufill®) and Vinyl Alcohols (Onyx®, ethylene vinyl alcohol (VA)) provided two unique solutions to the problems of previous embolic agents by offering shape-adaptable occlusion. NBCA forms an adhesive cast of the vessel wall but requires the simultaneous injection of the glue with water via catheter. This complicated application procedure requires a higher skill level to allow for some control over the aggregation process. Ethylene VA glues offer a more-user friendly approach by relying on concentration-dependent application timelines that use circulatory removal of solvent to produce an occlusive precipitate. While these newer materials offer valuable improvements over early techniques and improved outcomes, both are limited by toxic components, poor control over aggregation, and formulations best suited to the circulatory system.

Today, lymphatic embolization is accomplished with a variety of materials and there are no clear best-in class options. Common materials are fibrin glue like BioGlue®, Floseal® Hemostatic Matrix topical, Onyx® and Trufill®. The liquid to solid transitions of these materials makes them particularly well suited to blocking small perforations and vessels where leaks are hard to localize. Their ability to be rapidly applied in large volumes makes them a valuable solution for larger leaks with irregular morphologies as well. Despite their utility, most embolic agents are not radiopaque and require modifications to allow for precise application and fewer complications. Trufill®, a glue based on radiolucent cyanoacrylate, is often used in combination with ethiodized oil to allow X-ray guided embolization. Nevertheless, changes in the formulation of the glue to add radiopacity can also affect the cohesiveness and hardening timeline, demonstrating a material challenge for these technologies.

All embolization agents have disadvantages that are exacerbated by use in the lymphatic system as they are not designed for those conditions; i.e., there are no FDA approved products indicated for lymphatic embolization. Onyx®, Trufill®, fibrin glues, and others were all designed for vascular use. While off-label use is common, using a medical device in an anatomical region for which it was not designed can lead to poor results. For example, Onyx® consists of dissolved synthetic polymer in DMSO as noted above. The polymer precipitates out of solution as DMSO is carried away by circulating fluid. In the circulatory system, for which it was designed, 280 liters of blood circulate every hour. In contrast, the lymphatic system circulates 4-5 liters per day, which drastically delays the application time required for Onyx® and causes a higher than intended DMSO concentration when it is used in the lymphatic system. As a result, it risks respiratory distress, pulmonary edema, vasospasm and endothelial necrosis. DMSO also poses a risk to patients who have implants not tested for contraindication. Other synthetic sealants like Hystoacryl® and Trufill® n-BCA rely on in situ polymerization and have an unpredictable polymerization time, as noted below. These cyanoacrylate-based embolic agents used have also caused neurotoxicity, hepatotoxicity, and edema as a result of formaldehyde release. To avoid similar impacts on functionality and potentially harmful side effects, design criteria taken from the lymphatic system should be used to inform development of embolization technologies with better safety profiles.

Common embolization agents also have undesirable material properties. As referenced above, sealants like Hystoacryl® and Trufill® n-BCA rely on in situ polymerization and have an unpredictable polymerization window, whereby that this delayed chemical reaction can prevent the formation of a bolus of adequate volume, cause spread of the sealant beyond the intended location, and adhere the catheter tip to the vessel wall. Once in place, nBCAs do have a strong adhesive effect, but they also cause vascular damage and inflammation. Although this enhances the embolization process, it raises questions as to whether the mechanism is valuable for long-term patient health. Further, even though n-BCA is used as a permanent embolic agent, embolization sites can re-open over time, potentially damaging vessel walls without offering the long-term treatment promised. Onyx® (EVA) has slightly more desirable material characteristics, offering customizable concentrations for release timelines and a formulation that precipitates over time to allow for more control over delivery. Unfortunately, it also has higher complication rates. Onyx® has an 8.5% rate of catheter attachment to the vessel wall. Often this results in the catheter being left inside the patient to avoid causing more damage. As another example, fibrin glues can cause problematic secondary embolization, and have shown only variable success as a lymphatic sealant. This poor control over mechanical behavior Illustrates two material challenges faced by these technologies: solidification timelines, and controlled cohesiveness.

While present embolic agents have offered vastly improved outcomes over conservative treatment methods, they are imprecise systems with significant disadvantages. As such, an improved embolic agent would be well received in the medical arts.

### BRIEF SUMMARY

The present disclosure includes disclosure of the development of an embolic agent specifically tailored to avoid the shortfalls of existing technologies. This embolic agent leverages the thermo-responsive properties of poly(N-isopropylacrylamide) (PNIPAM). PNIPAM is a smart biostable polymer used for drug screening, biotechnology, and medical diagnostics applications. Through co-polymerization with other monomers, the thermo-responsive behavior and mechanical strength can each be tailored to create a hydrogel that form-fits upon injection, adapting to irregular margins and sealing lymphatic leaks. These properties can be further altered by the addition of chemical or physical cross-linking agents, or anionic additives that alter the phase transition. Through an iterative design process, this customizable behavior offers a viable chemical platform for many biomedical technologies. Further, simplified free radical production and end product stability will significantly lower the cost of this sealant in comparison to existing technologies. Through an iterative design process that tested both mechanical behavior and practical suitability, this research produced a novel formulation of lymphatic embolization agent (LEA) that relies on tailored thermo-mechanical hardening to safely and reliably occlude lymphatic vessels.

A sealant has been developed that improves upon current treatments in the following respects: 1) Optimized delivery window, 2) No in situ polymerization, 3) No harmful byproducts and 4) Cost effective formulation. During the design process, particular attention was given to materials that were tunable, safe, and effective occlusion agents. The thermo-responsive properties of poly(N-isopropylacrylamide) (PNIPAM) provided the ideal foundation for the development of an optimized lymphatic embolization agent (LEA). Through a combination of model-based and material testing, a hydrogel was developed that balances conformational factors to achieve a customized transition temperature, radiopacity suitable for visualization, mechanical properties suitable for delivery via 3 Fr catheter, sufficient cohesion once applied to resist migration under physiological pressures, and an improved safety profile. The material and benchtop results for this product demonstrate the suitability of this new hydrogel not only as a LEA, but for healthcare applications across internal medicine.

The present disclosure includes disclosure of an optimized lymphatic embolization agent (LEA), comprising a NAM hydrogel and tantalum at a mixture of at or about 1:3 tantalum to NAM hydrogel, wherein said LEA is radiopaque.

The present disclosure includes disclosure of an optimized lymphatic embolization agent (LEA), wherein the NAM hydrogel comprises N₉₃Am₇.

The present disclosure includes disclosure of an optimized lymphatic embolization agent (LEA), wherein the N₉₃Am₇ is produced by dissolving NIPAM, Am and 2,2′ azobisisobutryonitrile in tetrahydrofuran, degassing under elevated temperature, and isolating the resultant product, wherein the resultant product comprises N₉₃Am₇.

The present disclosure includes disclosure of an optimized lymphatic embolization agent (LEA), wherein the N₉₃Am₇ is synthesized by free radical polymerization.

The present disclosure includes disclosure of an optimized lymphatic embolization agent (LEA), configured for injection into a mammalian subject through a 3 Fr catheter.

The present disclosure includes disclosure of an optimized lymphatic embolization agent (LEA), configured to solidify within 45 seconds at a temperature of 37° C.

The present disclosure includes disclosure of an optimized lymphatic embolization agent (LEA), configured to solidify within 30 seconds at a temperature of 37° C.

The present disclosure includes disclosure of an optimized lymphatic embolization agent (LEA), configured to solidify within 15 seconds at a temperature of 37° C.

The present disclosure includes disclosure of an optimized lymphatic embolization agent (LEA), configured to resist displacement under 800 mmHg of pressure after 60 seconds at 37° C.

The present disclosure includes disclosure of an optimized lymphatic embolization agent (LEA), configured as a lymphatic sealant.

The present disclosure includes disclosure of a method of occluding a lymphatic vessel, comprising introducing an optimized lymphatic embolization agent (LEA) into a mammalian patient at or near a lymphatic vessel to occlude said vessel.

The present disclosure includes disclosure of a method of occluding a lymphatic vessel, wherein the step of introducing comprises introducing at or about 3 ml of the LEA so that at or about 1 ml of solid glue forms at or about 37° C.

The present disclosure includes disclosure of a method of occluding a lymphatic vessel, wherein the solid glue resists migration.

The present disclosure includes disclosure of a method of treating lymphatic leakage, comprising introducing an optimized lymphatic embolization agent (LEA) into a mammalian patient at or near a lymphatic vessel to treat the lymphatic leakage.

The present disclosure includes disclosure of a method of treating lymphatic leakage, wherein the step of introducing comprises introducing at or about 3 ml of the LEA so that at or about 1 ml of solid glue forms at or about 37° C.

The present disclosure includes disclosure of a method of treating lymphatic leakage, wherein the solid glue resists migration.

The present disclosure includes disclosure of methods to perform thoracic duct embolization, as shown and/or described herein.

The present disclosure includes disclosure of methods to perform a thoracic duct embolization procedure using materials suitable for use as a sealant, as shown and/or described herein.

The present disclosure includes disclosure of methods to perform a thoracic duct embolization procedure using NIPAM-based hydrogels suitable as a sealant, as shown and/or described herein.

The present disclosure includes disclosure of methods to perform a thoracic duct embolization procedure using embolization agents as a sealant, as shown and/or described herein.

The present disclosure includes disclosure of methods to perform a thoracic duct embolization procedure using hydrogels suitable as a sealant, as shown and/or described herein.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A shows an illustration of the application process for a temperature responsive lymphatic embolization agent to a lymphatic leak via intranodal catheter insertion, according to an exemplary embodiment of the present disclosure;

FIG. 1B shows temperature-sensitive solution dynamics of PNIPAM hydrogels, showing a transition from hydrophilic availability to collapsed globule state with stronger internal networking, according to an exemplary embodiment of the present disclosure;

FIG. 1C shows a dalia flower cast and molded PNIPAM hydrogel, demonstrating high-fidelity shape retention well-suited to gripping various features of vessels or tears in the lymphatic system as sealant, according to an exemplary embodiment of the present disclosure;

FIG. 1D shows deployment of a PNI Hydrogel at 34° C. through a temperature-controlled 3 Fr catheter with 0.46 mm internal diameter, according to an exemplary embodiment of the present disclosure;

FIG. 2A shows a structural comparison of NIPAM and co-Acrylamide Polymers highlighting H-bonding sites, according to an exemplary embodiment of the present disclosure;

FIG. 2B shows a storage modulus from strain ramp of 30% NAM hydrogel at temperatures above (40° C.) and below (20° C.) LCST, noting an increase by a factor of 10 despite continuous deformation, according to an exemplary embodiment of the present disclosure;

FIG. 2C shows a loss modulus from strain ramp of 30% NAM hydrogel at temperatures above (40° C.) and below (20° C.) LCST, noting an increase by a factor of 10 despite continuous deformation, according to an exemplary embodiment of the present disclosure;

FIG. 2D shows a deformation frequency ramp above and below LCST, noting that storage modulus increased by factors of 10-100 during continuous deformation, according to an exemplary embodiment of the present disclosure;

FIG. 2E shows a loss modulus increased by a factor of 10 to 100 during continuous deformation during a frequency ramp, according to an exemplary embodiment of the present disclosure;

FIG. 3A shows Polymer concentration effects on temperature sensitivity and LCST in PNI hydrogels, according to an exemplary embodiment of the present disclosure;

FIG. 3B shows a Confirmation of a designed LCST shift in the co-Acrylamide NAM hydrogel, according to an exemplary embodiment of the present disclosure;

FIG. 3C shows Hydrogen bonding sites provided more networking opportunities after a transition to the globule state above LCST, according to an exemplary embodiment of the present disclosure;

FIG. 3D shows Temperature dependent complex viscosity as determined by fixed temperature oscillatory rheology showING a 1,000,000-fold increase for the NAM hydrogel in comparison to only a 10-fold increase for the PNI hydrogen, according to an exemplary embodiment of the present disclosure;

FIG. 3E shows a NAM hydrogel moduli above (light) and below (dark) LCST confirm the strengthening of the hydrogel network by inversion from loss to storage modulus dominance, according to an exemplary embodiment of the present disclosure;

FIG. 4A shows a mixture of 30% NAM with tantalum at room temperature (mass ratios of 1:8, 1:4, and 1:2), according to an exemplary embodiment of the present disclosure;

FIG. 4B shows an additive-induced LCST decrease in the NAM hydrogel, according to an exemplary embodiment of the present disclosure;

FIG. 4C shows an injection of a 30% NAM and Tantalum (3:1) at 37° C. through a 3 Fr catheter. according to an exemplary embodiment of the present disclosure;

FIG. 4D shows a small volume LEA deployment under x-ray at room temperature, according to an exemplary embodiment of the present disclosure;

FIG. 4E shows an LEA benchtop pressure rig allowing for temperature-controlled testing of occlusive efficacy, according to an exemplary embodiment of the present disclosure; and

FIG. 4F shows a hydrogel candidate viscosity above (light) and below (dark) LCST confirm differences in material properties elicited by changes in monomers, concentration, and additives, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

### Determination of Mechanical Criteria

The transition from a malleable form to solid is a universal property of embolic agents. They each accomplish this mechanical transition slightly differently, but of chief importance for all of them is a reliable transition stimulus and window. In a clinical setting, the ability to produce a consistent behavior from an occlusive agent makes the difference between a catheter glued to a vessel wall and a procedure without any adverse events.

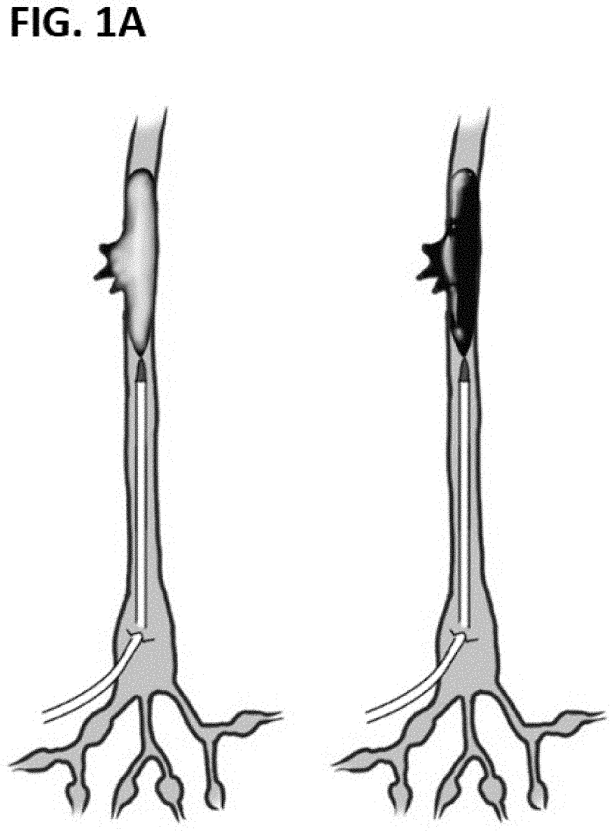

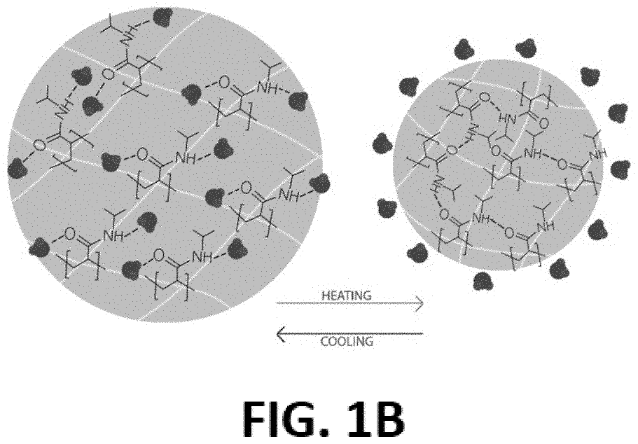

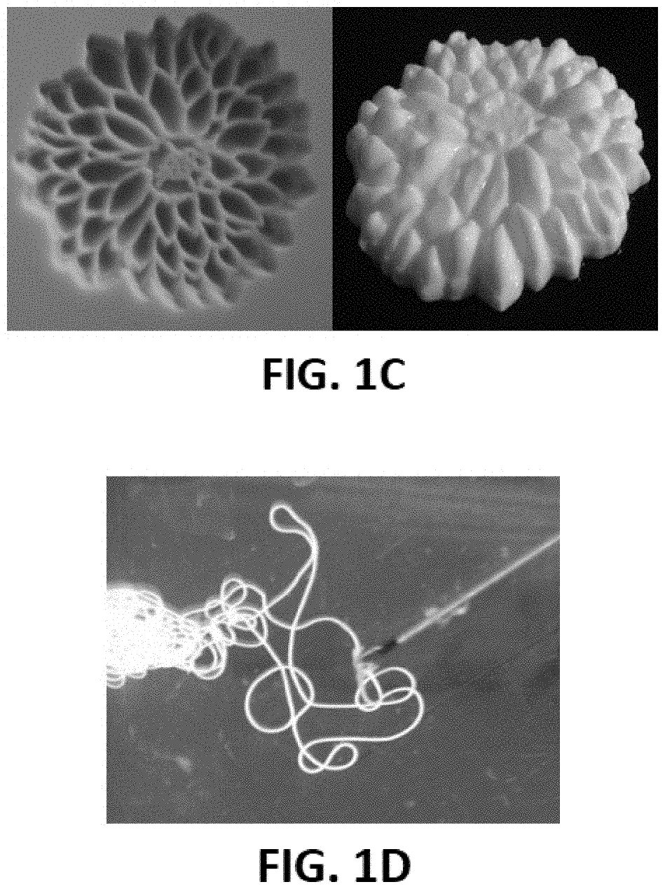

There are many responsive materials capable of producing this reliable change in behavior, but one of the most notable is poly(n-isopropylacrylamide) (PNIPAM). It is uniquely suited for the targeted deployment to a higher temperature region where aggregation is desired. Further, having been widely studied, it boasts a biocompatibility and customizability rarely observed with other synthetic molecules. PNIPAM was first synthesized in 1956 and was soon recognized as a smart, biostable polymer. It undergoes a reversible transition from a hydrophilic coil state to a hydrophobic globule state at a specified temperature known as the Lower Critical Solution Temperature (LCST) (such as shown in FIG. 1B). This unique molecular behavior results in different types of mechanical properties depending on the polymeric structure, concentration of that polymer in the hydrogel, and any other additives at play in the formulation. At higher concentrations, these hydrogels can have enough cohesion for shape retention with significant detail, facilitating occlusion of several vessel geometries and types by conformation to surface details (such as shown in FIG. 1C). Furthermore, by modifying molecular structures, both LCST and mechanical strength can be optimized for different applications.

NIPAM homopolymers are well understood, but their suitability for use as embolic agents has not been widely pursued. The primary criteria defining their suitability as embolic agents are their phase transition dynamics and the material properties on either side of the LCST. To isolate these parameters, a 20% (w/w) solution of PNIPAM (PNI), well-documented to have a phase transition at 32° C., was created to examine preliminary suitability for delivery via a 3 Fr catheter. A temperature-controlled benchtop model was used to test suitability for controlled deployment via catheter. A 40 cm of catheter was submerged in a temperature-controlled water bath to simulate a lymphatic embolization operation. Though a 3 Fr catheter with internal diameter 0.46 mm was not able to deploy the hydrogel at 37° C., a 4 Fr with a larger internal diameter (0.89 mm) was able to effectively deliver the hydrogel through the catheter (as shown in FIG. 1D). Running a cold saline solution prior to injection of sealant offered prolonged delivery timelines but provided inconsequential benefits during early attempts to imbue the hydrogel with radiopacity. A 1:1 mixture of that hydrogel with tantalum resulted in a glue that could not be deployed at bath temperatures above 34° C. This result demonstrated two necessary design inputs: an LCST between the PNI (32° C.) and body temperature (37° C.), and an LCST slightly higher than anticipated due to the premature solidification caused by the tantalum.

The ideal material properties for the sealant were also examined qualitatively. Embolic agents must provide long-term occlusion, resisting pressure, compression, and other forces for an indefinite timeframe. This task requires cohesive strength and durability not observed in PNIPAM hydrogels. These homopolymer hydrogels have a characteristic fragility that can be observed easily in the semi-solid state above LCST. These PNI hydrogels can easily be crumbled between fingers and are generally not suitable for biomedical sealants. Increasing concentration can help in this regard, but the limited ability to deploy even a 20% PNI hydrogel demonstrated that simply using a homopolymer would not work. Following these qualitative observations, the third design input of increased hydrogel strength was identified. In order to strengthen the material properties of the hydrogel, molecular modification or cross linkers must be used. Based on the previous work of Bayat et. al., comonomer selection has been shown to produce both the LCST shift and an increase in strength, providing a natural starting point.

### Comonomer Selection and Characterization

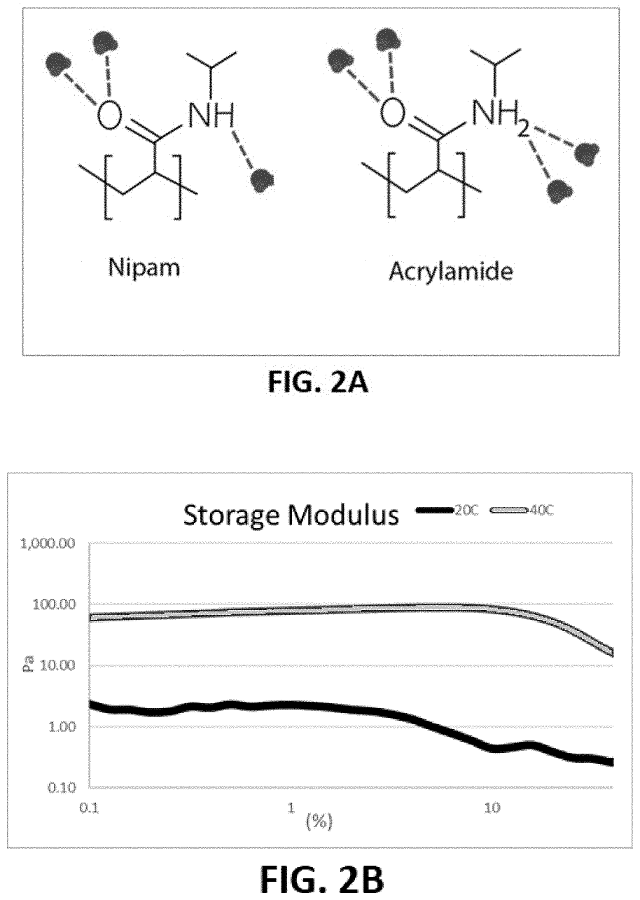

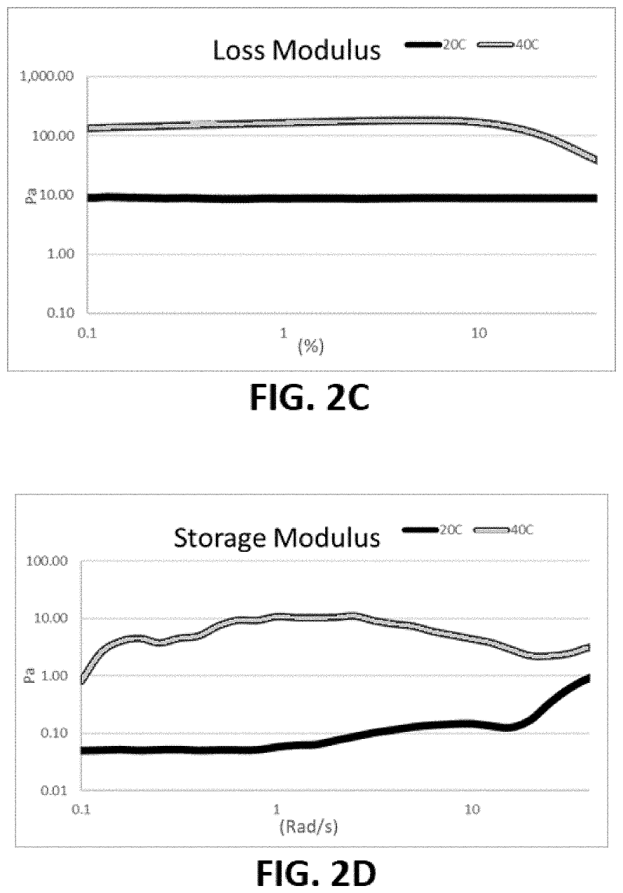

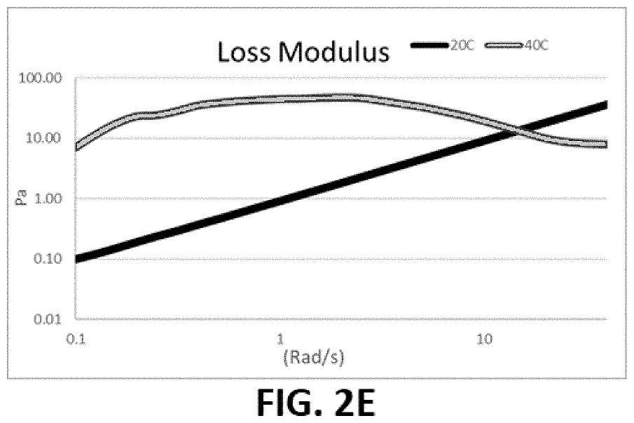

Initially, three hydrophilic comonomers were identified with co-monomers suitable for increasing the hydrogel LCST: Acrylamide, Acrylic Acid, and Methacrylic Acid. Acrylamide was chosen as the preferred candidate due to concerns about hydrogel pH. Further, a mouse toxicity assessment for a copolymer of NIPAM and Acrylamide has already shown preliminary biocompatibility and safety. These traits offered a promising first LEA attempt and so a N₉₃Am₇ (NAM) (by mass) conformation was synthesized by Free Radical Polymerization. In comparison to PNI homopolymers, this copolymer contains a greater proportion of hydrophilic subunits, but also provides more hydrogen bonding sites by way of the dual hydrogens on the amide group. This increased capacity for supramolecular linkage has been linked to the formation of stronger gel networks (FIG. 2A). Though PNI concentration of 20% polymer was used, a qualitative assessment of viscosity below LCST showed the viscosity of a 30% polymer NAM hydrogel to be a closer approximation. This disparity can be attributed to differences between the hydrogel poly dispersities and molecular weights. High Mn samples generally correspond to higher viscosities. Since the PNI homopolymer was purchased from Sigma Aldrich and has a published Mn of 40,000, it can be assumed that NAM polymer has a lower Mn. This sample was then characterized and benchmarked to the PNI sample to determine the optimal transition temperature, solution concentration, and rheological characteristics. Benchtop testing was also used as a preliminary determination of suitability and practical assessment of phase change timeline.

To assess the viability of the NAM hydrogel, oscillatory rheology was performed to record gel responses across a range of strains and temperatures. Overall mechanical properties were then observed in the Loss (G″) and Storage (G′) moduli, quantifications of viscous and elastic behavior. These characteristics play a significant role in LCST-triggered phase transitions. Prior to gelation, a loss modulus that is too high can indicate a material that is too viscous and unable to be easily injected. At higher temperatures, a storage modulus that is too low indicates that the material is not sufficiently cohesive and may deform or break under physiological pressures. The comparison of these two indicators can provide further clues about supramolecular structure. A dominant loss modulus is characteristic of a primarily viscous fluid. On the other hand, a dominant storage modulus occurs when the material resists deformation, a sign of material strength and molecular networking. This strengthening can be observed in frequency and strain sweeps applied to a 30% NAM hydrogel. At temperatures above (40° C.) and below (20° C.) LCST, the amount (amplitude) and frequency of strain were increased to determine the critical points and regions of linearity for hydrogel analysis. At 40° C. above the LCST, tight hydrogel structure gave way to very clear regions of linearity and critical points of structural breakdown around 10% strain (See FIGS. 2B and 2C). Below the LCST, a linear region up to 5 rad/s was observed (as shown in FIGS. 2D and 2E). Given the interest in behavior across a range of temperatures, overlap in regions of linearity was of critical interest. Parameters of 1% strain and 1 Rad/s were chosen for further analysis.

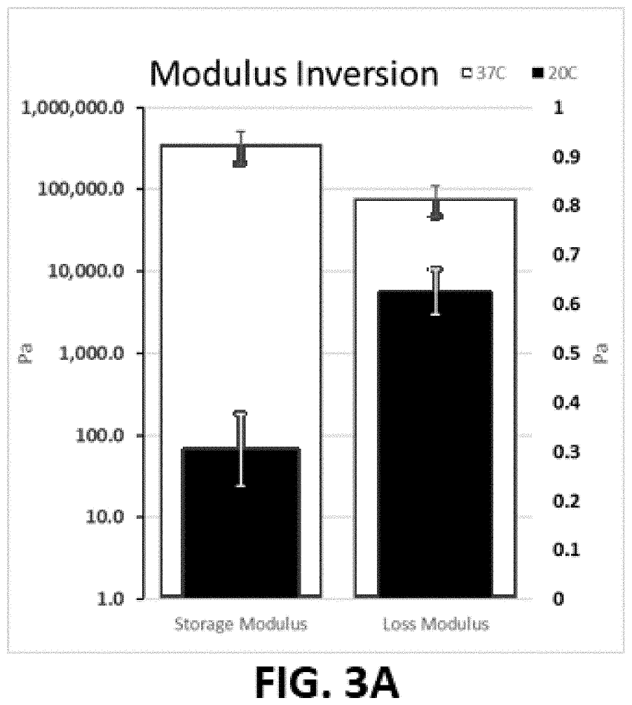

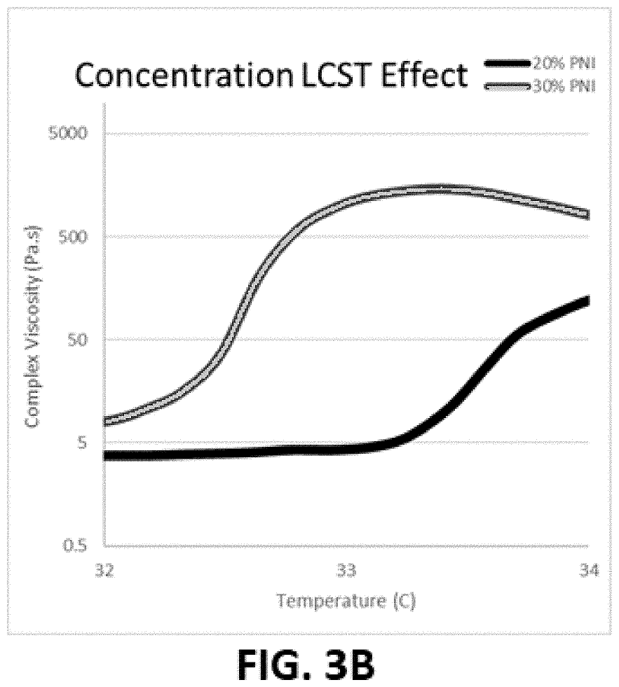

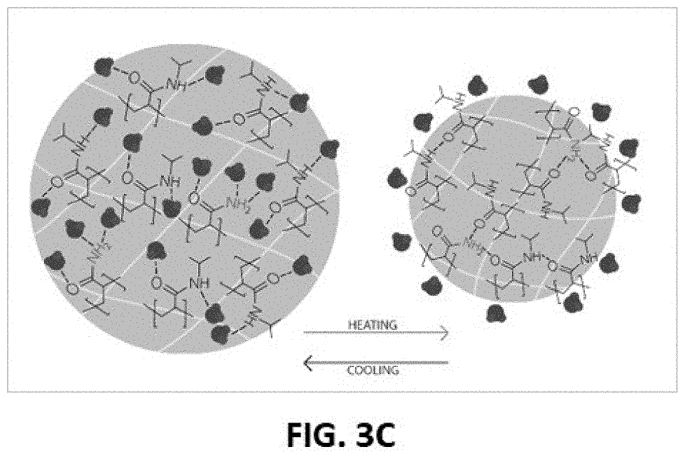

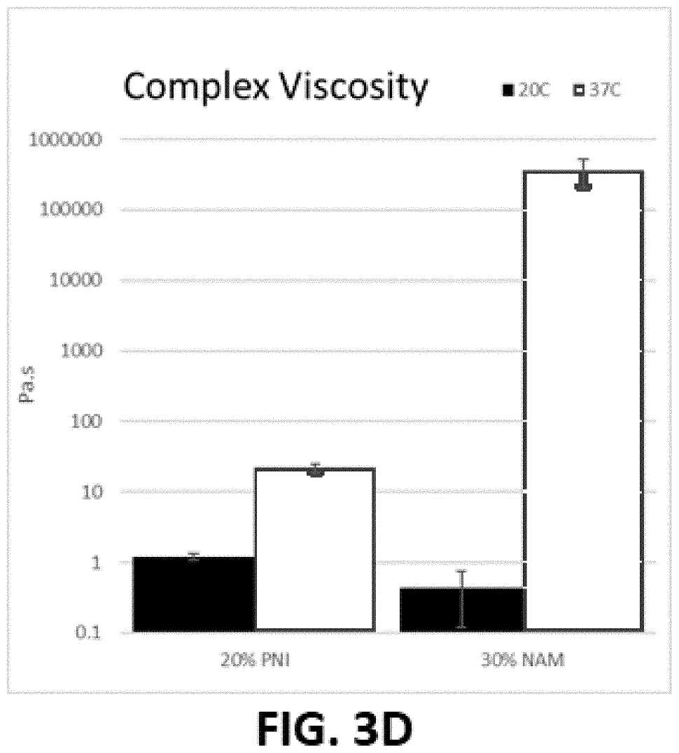

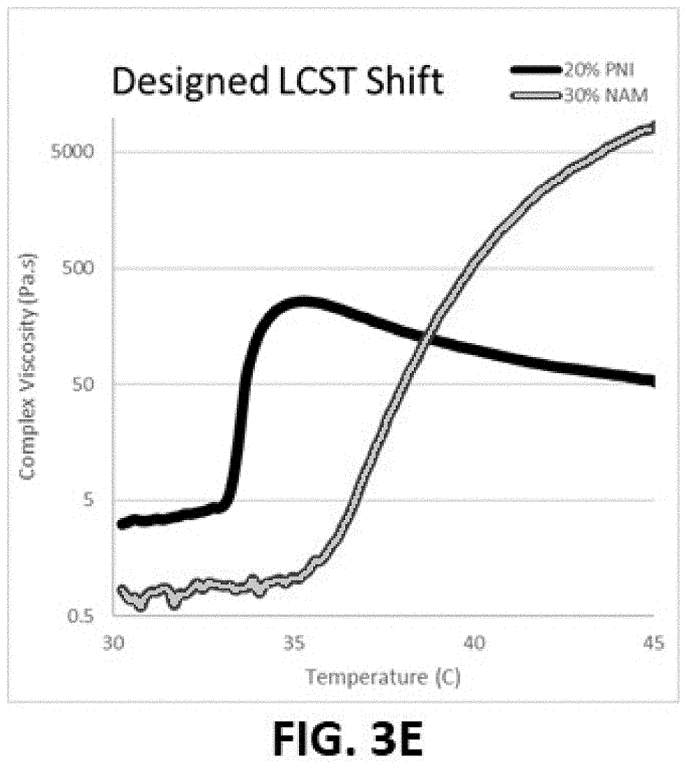

Temperature sweeps were conducted to determine the effects of concentration and structural modification on the phase transition. Previous research has already demonstrated an inverse relationship between polymer concentration and hydrogel LCST, and a direct relationship between polymer concentration and hydrogel material strength (viscosity). Our results using a comparison of two PNI hydrogels with different concentrations confirmed these earlier findings and further illustrated the need for precise tuning of hydrogel behavior (as shown in FIG. 3A). A comparison of viscosity as temperature was increased across the LCST also confirmed the successful increase of hydrogel LCST through copolymerization (˜36°) (see FIG. 3B). Both the homopolymer and NAM hydrogels exhibited characteristic indifference to temperature, below LCST, with constant complex viscosity. Once the molecular networks start to form however, a peak is quickly observed for both samples that reflects an increase in overall resilience on the order of 100 and 10,000 for PNI and NAM hydrogels, respectively. This significant difference indicated that the hydrogel LCST was not only significantly altered, but the mechanical strength of this LEA candidate vastly exceeded that of our benchmark.

Further examination of the NAM hydrogel mechanical properties indicated that an almost ideal candidate was found for the LEA. Complex viscosity comparisons during temperature ramp or earlier strain/frequency sweeps constantly deformed the sample but still demonstrated a significant increase in the strength of the gel. Before the transition however, the LEA hydrogel was noted to have a much lower viscosity despite having 150% of the PNI benchmark's concentration. It was theorized that the acrylamide co-monomer played two roles in this regard. By offering more hydrogen bonding sites, the copolymer was more hydrophilic, more readily dissolving below LCST. Above LCST, however, those hydrogen bonding sites provided more networking opportunities after a transition to the globule state (as shown in FIG. 3C). Oscillatory time sweeps at fixed temperatures confirmed these molecular interactions by not deforming the sample until the phase change was complete. The results indicated that the 30% NAM hydrogel increased in complex viscosity by a factor of almost 1 million when heated to 37° C. In contrast the 20% PNI sample only increased by a factor around 20 (as shown in FIG. 3D). This difference is well suited to delivery via catheter, but also underscores the significant networking that occurs during the NAM hydrogel phase transition. This increase in strength was further confirmed by a comparison of the storage and loss moduli across the LCST (see FIG. 3E). At 20° C., loss modulus slightly overcame storage modulus, but both were negligible. After transition, however, both moduli increased and the storage modulus dominated, indicating significant cohesiveness and formation of strong molecular networks. The increase of storage modulus by a factor of over 1 million further reinforces that conclusion. Acrylamide subgroups have a tendency to form hydrogen bonds with themselves, as is evidenced by their tendency to form poly-molecular aggregates in solution. The NAM hydrogel leverages that behavior to provide both the strength and LCST suitable for a LEA.

### LEA Optimization

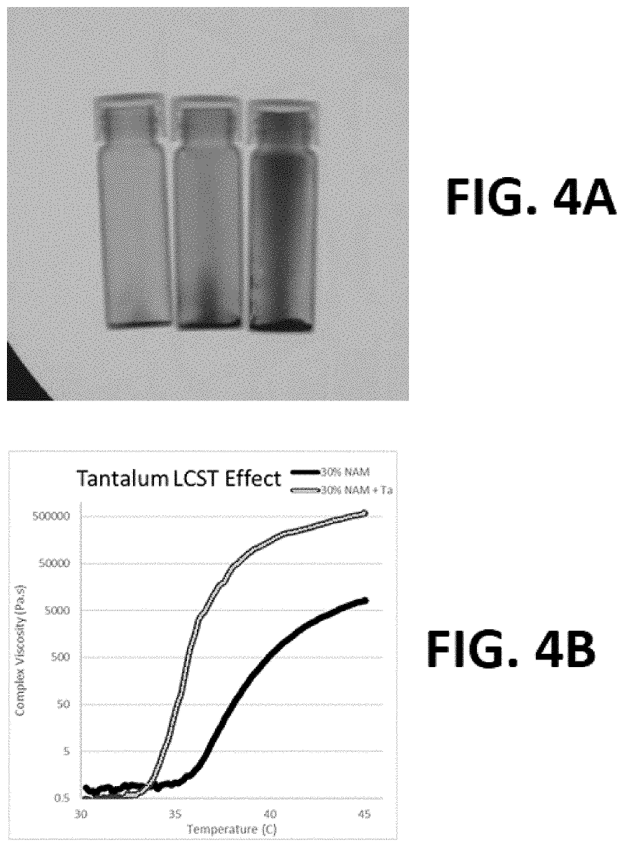

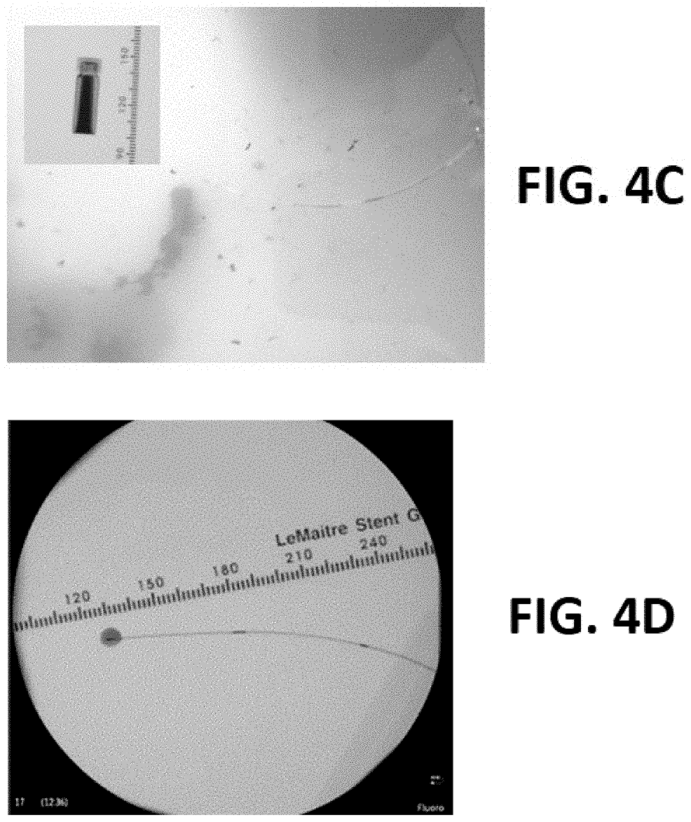

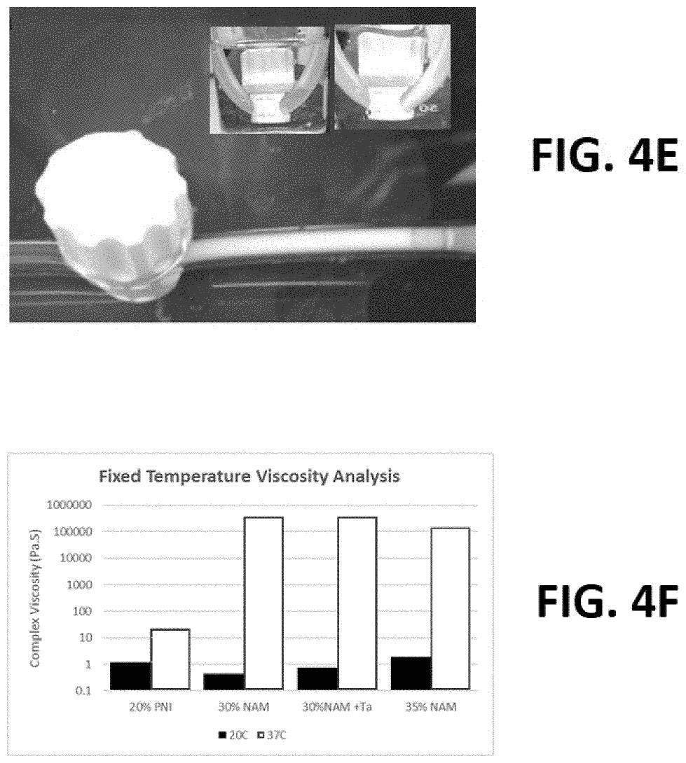

The cohesiveness of the NAM hydrogel established its suitability for use as a preliminary LEA. Even so, it still lacked radiopacity. This presented a potential design challenge as the addition of various additives to NIPAM hydrogels can vastly alter their behavior and strength. Tantalum, a well-known radiopaque element that can be mixed with sealants proved to only slightly alter hydrogel material properties. Ratios of 1:8, 1:4, and 1:2 tantalum to 30% NAM hydrogel were created to determine the optimal formulation (FIG. 4A). Varying levels of radiopacity were obtained and weighed against the other effects of tantalum in solution. In a qualitative assessment of PNI hydrogels, adding tantalum reduced the temperature at which it could be deployed via catheter from 37° C. to 34° C. Following tantalum addition to the 30% NAM hydrogel, a clear lowering of the LCST by 2° C. to around 34° C. was observed, confirming that effect (FIG. 4B). 1:3 tantalum to NAM hydrogel mixture was chosen to limit the cancellation effect of the tantalum on the purposefully increased LCST while enabling surgical visualization.

### Benchtop Validation

A combination of controlled temperature catheter injection and pressure testing allowed for a rapid determination of design inputs. While the 20% PNI sample was suitable for catheter delivery at 37° C. and could withstand 4× maximum lymphatic pressure (ave:200, test:800 mm Hg), the transition occurred to quickly for the gel to be useful. Further, concerns were raised about the overall cohesivity of the hydrogel for a long-term use case.

To validate the LEA candidate, more benchtop proof-of concept testing was performed. In greater detail, LEA was injected through 3 Fr catheter, 40 cm of which was at a range of controlled temperatures of 37° C. This provided preliminary determination of suitability for deployment in a simulated use case. PNI, NAM, and NAM+Ta hydrogels were all tested using this method. These tests were useful for determining target concentration and formulation viability. It took 10-15 sec to deliver 1.0 ml of NAM and solidification took place within 15-20 sec (FIG. 4C). One ml of solid glue is generally sufficient to plug the lymphatic vessel. Deployment under Xray was also performed to assess the effective visibility in a clinical setting (FIG. 4D). Preliminary efficacy was assessed by pressure testing. LEA was injected into a 5 mm diameter plastic tube, which was then placed in a thermal bath of 37° C. for 60 s and put under pressure (800 mm Hg) (FIG. 4D). These procedures were used for exploratory testing and then repeated with N=3 for the final 30% NAM prototype. The amount of the NAM and NAM+Ta hydrogels was sufficient to both plug the vessels and reliably resist displacement at 37° C. under 800 mmHg of pressure.

Results from these tests also reinforced material analysis. At temperatures below LCST, NAM hydrogel viscosity increased slightly as result of tantalum addition. At higher temperatures, however, there were no significant differences between viscosity or moduli, indicating that the tantalum did not have any adverse effects on hydrogel cohesion (FIG. 4E). After the successful deployment and occlusion tests, this further confirmed the suitability of tantalum for incorporation into NIPAM hydrogels. The slight variations in behavior between the samples and many variables at play do present large opportunities for continued work.

The successful occlusion of a simulated vessel indicated that the LEA identified herein is suitable for validation in biological models, but significant work must be performed to fully characterize the material and establish suitability. Long-term shape retention and degradation timelines must be studied. Additionally, the impact of any unreacted monomer on the hydrogel as a cross linking agents should be explored. Tolerances for material suitability will also need to be established via robust methods to ensure long-term efficacy without adverse effects.

### Materials and Methods

### Homopolymer Source and Copolymer Synthesis

Poly(N-isopropylacrylamide) homopolymer Mn=40 k was purchased from Sigma Aldrich for initial hydrogel preparation. Copolymers of NIPAM and Acrylamide (Am) were synthesized using free radical polymerization. For N₉₃Am₇, a solution of NIPAM (9.3 g), Am(0.7 g) and 2,2′ azobisisobutyronitrile (10 mg) were dissolved in tetrahydrofuran (THF). The solution was then degassed under nitrogen and heated to 55° C. for 24 hours. Copolymer product was isolated using hexanes, filtered, washed, and dried under vacuum. Target monomer ratios were derived from literature and information on copolymers that may be purchased from common chemical vendors.

### Rheological Analysis

A TA Instruments DHR 2 Rheometer was used to measure the rheological properties of the hydrogels. A 0.15 ml of hydrogel was injected via syringe onto a temperature-controlled plate. Strain-Amplitude and Frequency sweeps were used to identify linear regions and critical points for the hydrogels at temperatures below (20° C.) and above (40° C.) LCST. Amplitude sweeps for parameter determination were performed with a frequency of 10 rad/s. Angular frequency sweeps were performed with an amplitude of 1% strain. After linear regions were identified, strain of 1% and frequency of 1 rad/s were used for all subsequent testing. Temperature ramp experiments were performed with a ramp rate of 1° C./min, spanning 30° C. to 45° C. Fixed temperature oscillatory tests were performed at fixed temperatures of 20° C. and 37° C. (body temperature). All experiments use a 20 mm parallel plate (0°) fitted with a solvent trap, 500 μm gap, and 2-minute temperature incubation period. N=3 for all rheological experiments. Samples tested include 20%, 30% PNI, 30% NAM, and 30% NAM+tantalum.

### Discussion

This work demonstrated that a NIPAM-based hydrogel has the potential for use as an internal embolic agent. By tailoring molecular structure, concentration, and additives an appropriate prototype was developed that showed sufficient durability and cohesion to withstand a basic model of a lymphatic use case. Structural modifications with acrylamide were sufficient to cause mega-fold increases in hydrogel viscosity, inviting many avenues of future research into the molecular dynamics that cause this significant supramolecular alteration. This work was initially motivated by the shortfalls of existing lymphatic embolization technologies. There are no sealants approved for this indication the resulting shortfalls of off-indication use have placed an unreasonable burden on patients. The drastic improvements in material properties and early validation data suggest that this technology could alleviate that burden.

### Functional Requirements for LEA

A goal of the studies performed herein was to overcome the mechanical, biocompatibility, and delivery shortcomings of existing technologies while meeting standard requirements for efficacy. The results presented here demonstrate a temperature-responsive sealant that can be injected as a low-viscosity liquid via 3 Fr catheter to a site of lymphatic leakage, and then undergo a heat-induced solidification to create a form-fitted, non-biodegradable occlusion. This blockage can then prevent the loss of fluid to interstitial tissue and restore equilibrium to the circulatory systems of the body (FIG. 1A). In a clinical setting, this technology would provide interventional radiologists with a valuable alternative to existing embolic agents through safer materials that are easier to handle. The developed glue material is novel in that it 1) has a phase transition specifically tuned for delivery via catheter, 2) is pre-polymerized yet offers significant cohesion to occlude lymphatic vessels, 3) balances radiopacity with hydrophilicity to have an optimized solidification window, 4) does not introduce chemicals with harmful side effects into the body, 5) will significantly lower the price of embolization treatments, and 6) will be the first embolization agent designed specifically with the lymphatic system in mind. The results presented offer proof of concept and characterization intended to demonstrate the viability of this formulation. Significant further testing will be needed to fully develop a viable medical product.

The LEA described in this report is the first hydrogel deployed to the lymphatic system as a sealant. Initial studies show that it takes 30-45 s to deliver the desired glue amount (3 ml is generally used to plug the lymphatic vessel which results in 1 ml of solid glue) through a 3 Fr catheter. A desirable goal is to complete the injection in less than 1 min with the hydrogel-imaging agent mixture through a 3 Fr catheter. Preliminary studies have also shown that a rapid transition to a shape-responsive ultra-cohesive material can ensure a stationary plug after the injection. The LEA described also resisted maximum lymphatic pressures with adequate opacity to be injected and monitored via radiography. Several experimental use cases have shown suitability of linear NIPAM copolymer hydrogels for use in medical devices. This study adds one more example of how molecular structure can be tailored to biomedical function.

The need for a smart alternative to existing technologies is further shown by the market size. The global transcatheter embolization and occlusion devices (TEO) market size was valued at $2.69 billion in 2016 and is expected to reach over $4.75 billion by 2022, according to a study by Grand View Research, Inc. The market has witnessed tremendous growth over the last decade. Paradigm shifts from clipping to coiling to liquid sealants have stimulated the demand for transcatheter embolization devices. A key driver of the market is the adoption of minimally invasive surgeries. Even so, costs have become a significant roadblock. Medical adhesives like cyanoacrylate, fibrin, and newer synthetics are significantly more expensive than conventional surgical technologies due to production costs. Stringent regulatory approval procedures, and the need for experienced healthcare professionals to perform TEO procedures are some factors that impede market growth. High costs for every type of embolization method are often cited as a disadvantage. By eliminating the need for complicated manufacturing processes, not requiring cure-resistant storage, and using only a few readily accessible components the preferred hydrogel(s) offer not only a competitive technical profile, but competitive overall costs.

The first set of experiments with the first generation of the glue has been promising. Future work will be to develop the second generation sealant, which would include chronic and acute disease swine testing, refining the sealant product, developing a commercialization plan, formal quality control with validation, GLP animal studies, and submission for an IDE first-in-man study. Future work on these technologies will also require examination of scalability. The simplicity of the manufacturing process offers a batch approach, but significant testing will still be required to show long term stability, suitably low levels of residual solvents and monomers, and reliable material properties. Should these various metrics be obtained with suitable results, it is anticipated that the technology will have many other biomedical applications that will be readily attainable with the map of chemical variables gained via this research.

While various embodiments and methods have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An optimized lymphatic embolization agent (LEA), comprising a NAM hydrogel and tantalum at a mixture of at or about 1:3 tantalum to NAM hydrogel, wherein said LEA is radiopaque.

2. The LEA of claim 1, wherein the NAM hydrogel comprises N₉₃Am₇.

3. The LEA of claim 2, wherein the N₉₃Am₇ is produced by dissolving NIPAM, Am and 2,2′ azobisisobutryonitrile in tetrahydrofuran, degassing under elevated temperature, and isolating the resultant product, wherein the resultant product comprises N₉₃Am₇.

4. The LEA of claim 2, wherein the N₉₃Am₇ is synthesized by free radical polymerization.

5. The LEA of claim 1, configured for injection into a mammalian subject through a 3 Fr catheter.

6. The LEA of claim 1, configured to solidify within 45 seconds at a temperature of 37° C.

7. The LEA of claim 1, configured to solidify within 30 seconds at a temperature of 37° C.

8. The LEA of claim 1, configured to solidify within 15 seconds at a temperature of 37° C.

9. The LEA of claim 1, configured to resist displacement under 800 mmHg of pressure after 60 seconds at 37° C.

10. The LEA of claim 1, configured as a lymphatic sealant.

11. A method of occluding a lymphatic vessel, comprising:
introducing the LEA of claim 1 into a mammalian patient at or near a lymphatic vessel to occlude said vessel.

12. The method of claim 11, wherein the step of introducing comprises introducing at or about 3 ml of the LEA so that at or about 1 ml of solid glue forms at or about 37° C.

13. The method of claim 11, wherein the solid glue resists migration.

14. A method of treating lymphatic leakage, comprising introducing the LEA of claim 1 into a mammalian patient at or near a lymphatic vessel to treat the lymphatic leakage.

15. The method of claim 14, wherein the step of introducing comprises introducing at or about 3 ml of the LEA so that at or about 1 ml of solid glue forms at or about 37° C.

16. The method of claim 14, wherein the solid glue resists migration.
